(19) United States
(12) Patent Application Publication
(10) Pub. No.: US 2003/0093105 A1
(43) Pub. Date: May 15, 2003

(54) Guide catheter for introduction into the subarachnoid space and methods of use thereof

(75) Inventor: Andrew Huffmaster, Fremont, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/328,373

(22) Filed: Dec. 23, 2002

Related U.S. Application Data
(63) Continuation-in-part of application No. 09/905,670, filed on Jul. 13, 2001.

(51) Int. Cl. A61M 29/00

(52) U.S. Cl. 606/192; 604/101.01

(57) ABSTRACT

Methods and devices for providing a medical device for use within the subarachnoid space are presented. The medical device may be a guide catheter including anchoring members or other mechanisms for securing a location or a pathway within the subarachnoid space. In some embodiments, the medical device may include multiple articulating elements which may pass through one another, with one or more of the articulating elements including an anchoring member.

### REFERENCE TO RELATED APPLICATIONS

This is a continuation-in-part of co-pending application Ser. No. 09/905,670 filed Jul. 13, 2001 entitled METHODS AND APPARATUSES FOR NAVIGATING THE SUBARACHNOID SPACE, which is expressly incorporated herein by reference.

### BACKGROUND

During the 20th century, brain neurosurgery has advanced via the introduction of microsurgical techniques, the development of new tools such as aneurysm clips, and the description of new operative approaches. Surgeons have developed elegant mechanisms to remove parts of the bones making up the skull (craniotomy) and operate on structures deep within the brain while attempting to minimize complications relating to the approach. The surgical approach to the intracranial and spinal subarachnoid space has historically included skin incision, dissection to either the cranium or spinal bony covering, removal of some bone, and dissection through the meninges to gain access to the neurological structures. While imaging modalities became integrated into diagnostic evaluations, only at the end of the last century were significant attempts made to integrate computed tomography, angiography, and most recently magnetic resonance (MR) scanning into the actual surgical procedures.

Unfortunately, craniotomy has limited the applicability of some present imaging modalities because the surgeon cannot simultaneously stand at the patient's head to operate on the brain via craniotomy, maintain sterility, and scan the brain using a large scanning apparatus that requires the patient to be held within it. There are limits to the ability to conveniently perform such surgery using currently-available imaging devices due to a conflict between the devices for acquiring images and the methods of operating on the brain.

An additional concern is that, while the brain surface is readily accessed via conventional craniotomy, the approach to deeper structures is progressively more difficult. The brain is often retracted after the craniotomy to facilitate access to different areas in and around the brain, and in some cases there is the need to remove brain tissue to gain access. Both retraction and removal create potential problems with maintaining sterility and avoiding direct injury to tissue, as well as the problem of putting tissue back into place without causing injury.

During the last 20 years, the development of endovascular neurosurgery has resulted in the creation of specialized devices for applications within arteries. These devices include not only catheters and guidewires, but also embolic materials that can be introduced via catheters, thereby enabling the enhancement of some procedures that are performed via craniotomy following embolization. In some cases, the need for craniotomy has been eliminated. However, access is limited to that achieved from within blood vessels.

The subarachnoid space is a compartment that contains the body of the spinal cord and cerebrospinal fluid (CSF). The CSF is a fluid that fills and surrounds the ventricles and cavities of the brain and the spinal cord, and acts as a lubricant and a mechanical barrier against shock. It is proposed that access to areas of the spinal cord and even the brain (intracranial space) may be gained by accessing the subarachnoid space. The access may include catheterization that may be used for diagnostic and therapeutic purposes. Several methods for accessing the subarachnoid space and applications for doing so, along with devices useful in such practices, are discussed in copending application Ser. No. 09/905,670 filed Jul. 13, 2001 entitled METHODS AND APPARATUSES FOR NAVIGATING THE SUBARACHNOID SPACE, which is expressly incorporated herein by reference.

As presented herein, several problems which may be encountered in percutaneous intraspinal navigation through the subarachnoid space arise in part from the physical structure of the subarachnoid space, which differs significantly from that of the vasculature. One difficulty is a lack of well-defined pathways. A second may be the significant number of obstacles within the subarachnoid space. For example, spinal nerves proliferate outward from the spinal cord, and can impede catheter progress while also presenting a delicate structure that should be traversed gently.

In several potential applications of intraspinal navigation, it may be desirable to provide a fluid infusion or drainage. For example, the CSF may be filtered to remove blood after a traumatic injury; one method of such filtration could be to remove the CSF at one location, pass it through a filter, and then infuse the filtered CSF back into the subarachnoid space. However, devices introduced to the subarachnoid space will typically have rather small lumens for fluid passage. Much as a garden hose will twist and move erratically when water is forced through it quickly, so may a catheter used in a fluid flush move and jerk erratically, causing unwanted displacement of the catheter tip. Hence it may be useful to not only secure a path through the subarachnoid space, but also to secure one or more specific positions therein.

### SUMMARY OF SEVERAL EMBODIMENTS

At least some embodiments include several solutions to these difficulties. Several embodiments provide a medical device, for example a catheter, for use in navigation of the subarachnoid space. In several embodiments, the medical device includes one or more anchoring devices. In one such embodiment, an anchoring device is used to create an anchoring point, which can facilitate or enhance other procedures. The anchoring device may be introduced as part of a medical device, such as a guide catheter, allowing other devices to pass therethrough, or it may be included as part of a device devised for other uses as well.

In several embodiments, there are multiple anchoring devices provided on one medical device. In one embodiment, a medical device is advanced so that a proximal-most anchoring device reaches a desired location, and a first anchoring device is then caused to perform its anchoring function, after which the medical device is manipulated to cause a next-most-proximal anchoring device to reach a second desired location and a second anchoring device is then caused to perform its anchoring function. Any number of anchoring devices may be included in various embodiments.

In another embodiment, an articulating medical device is used, where multiple components overlap one another. An outermost component may include a first anchoring device. The medical device may be advanced until the outermost component is at a desired location, and the first anchoring device may then be caused to perform an anchoring function; additional components including additional anchoring devices may be provided slidably disposed within the outer component and adapted to extend beyond the distal end of the outer component. By providing successively more distal anchoring devices, a path for entry into and passage through the subarachnoid space may be defined for example, for introducing a therapeutic or diagnostic device. Further, a stabilizing passage or point may be provided, for example, to assist with procedures where a detrimental or erratic motion of the distal end of the device is anticipated. In other embodiments, the medical device including one or more anchoring members may be directly used or may include a therapeutic or diagnostic apparatus or device.

In some embodiments, the anchoring members are inflatable devices, and the components or devices including them may include inflation lumens. In other embodiments, anchoring members are provided including shape memory materials, and the components or medical devices including them may include heating or cooling devices to cause actuation of the shape memory materials. In still other embodiments, the anchoring members may include retractable engagement members that may be caused to engage surrounding tissue by application of a pushing or pulling force, or by withdrawal or advancement of a covering sheath, for example.

In additional embodiments, an anchoring function may be effected by including variable stiffness elements. For example, in one embodiment, a flexible medical device such as a guide catheter may be introduced into the subarachnoid space, the medical device including several lumens. A stiffener may be introduced into one of the several lumens, the stiffener being chosen so that as the stiffener is inserted into the medical device, the medical device becomes more rigid. In some embodiments, the stiffener may be heated before introduction into the medical device to make the stiffener more flexible, and as the stiffener cools, the medical device is made more rigid.

### BRIEF DESCRIPTION OF THE DRAWINGS

FIG. 1A is a diagrammatic side view of an entry into a spinal subarachnoid space with an example guide catheter having an anchoring member;

FIG. 1B is a cross sectional view of the example embodiment guide catheter of FIG. 1A at a location corresponding to the anchoring member;

FIG. 2 is a schematic side view of an illustrative embodiment showing a guide catheter;

FIGS. 3A-3C are cross sectional views of portions of the example guide catheter shown in FIG. 2;

FIGS. 4A-4C are schematic side views of an illustrative embodiment showing a guide catheter in several stages of placement;

FIG. 5 is a cross sectional view of a proximal portion of the example guide catheter shown in FIGS. 4A-4C;

FIG. 6 is a schematic side view of a proximal portion of the example guide catheter shown in FIGS. 4A-4C;

FIGS. 7A-7E are cross sectional views of sections of an illustrative guide catheter;

FIG. 7F is a schematic side view of an illustrative guide catheter corresponding to the example embodiment illustrated throughout FIGS. 7A-7E;

FIGS. 8A-8B are schematic side views of an illustrative guide catheter including an anchoring member in retracted and deployed positions;

FIG. 9 is a schematic side view of an illustrative guide catheter having multiple anchoring members;

FIGS. 10A-10B are schematic side views of another illustrative guide catheter including an anchoring mechanism in retracted and deployed positions;

FIG. 11 is a schematic side view of another illustrative guide catheter with an anchoring member covered by a membrane;

FIGS. 12A-12B are schematic side views of another illustrative guide catheter including a shape memory anchoring mechanism; and

FIG. 13 is a diagrammatic side view of an entry into a spinal subarachnoid space with an example guide catheter having an anchoring member and including an entry sheath.

### DETAILED DESCRIPTION OF SOME EMBODIMENTS

The following detailed description should be read with reference to the drawings. The drawings, which are not necessarily to scale, depict illustrative embodiments and are not intended to limit the scope of the invention.

As used herein, the term “about” applies to all numeric values, whether or not explicitly indicated. The term “about” generally refers to a range of numbers that one of skill in the art would consider equivalent to the recited values (i.e. having the same function or result). In many instances the term “about” may include numbers that are rounded to the nearest significant figure.

Also as used herein, the term anchoring function is descriptive of the function of providing a resistance to movement. An anchoring function need not be a permanent or complete resistance to movement, and it may be directionally sensitive. For example, a horizontal anchoring function need not limit axial rotation nor movement in a vertical direction. Thus, to perform an anchoring function is to impede or make more difficult movement in at least one direction.

While many of the embodiments described herein are described in terms of potential uses in the subarachnoid space and even the intracranial space, many of these embodiments may also find use in catheterization or intervention in other areas of the anatomy, including but not limited to the digestive tract, the vasculature, the lungs, other soft tissues, etc. Further, while much of the following description includes references to human anatomy, other vertebrate organisms sharing some skeletal similarity to humans may be amenable to methods and devices such as those disclosed herein. One example would be use of methods and devices for introduction into the subarachnoid spaces of animals having a skeletal structure defining such spaces. Thus, for example, in some embodiments of methods and devices may be used to access the subarachnoid space of other vertebrate organisms including mammals, birds, reptiles, fish or amphibians. Some methods or devices may be useful, for example, in veterinary procedures.

In the embodiments shown in the Figures, the medical device is depicted as a guide catheter. However, the invention is not intended to be limited to a guide catheter. It should be appreciated that the device could be any medical device designed to pass through an opening or body lumen. For example, the device may comprise another type of catheter (e.g., therapeutic, or diagnostic catheter), guidewire, endoscopic device, laproscopic device, an embolic protection device, and the like or any other such device. Several example applications, methods, and devices for use in the spinal and intracranial subarachnoid spaces are noted in co-pending application Ser. No. 09/905,670 filed Jul. 13, 2001, entitled METHODS AND APPARATUSES FOR NAVIGATING THE SUBARACHNOID SPACE, which is incorporated herein by reference.

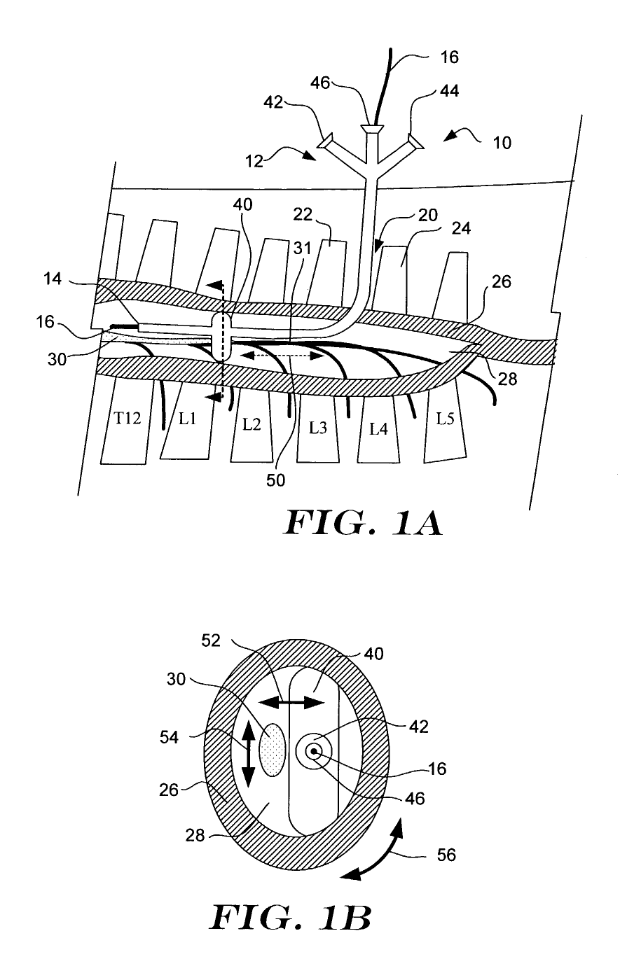

FIG. 1A is a diagrammatic side view of an entry into a spinal subarachnoid space with a medical device, which in this example is a guide catheter having an anchoring member. Guide catheter 10 includes a proximal end 12 and a distal end 14. Guidewire 16 extends through a lumen in guide catheter 10. The guide catheter 10 passes through interspace 20 between bony structures 22, 24, through dural membrane 26 and into spinal subarachnoid space 28, which contains spinal cord 30 and spinal nerves 31. For the illustrative embodiment shown, the interspace 20 passed through is in the lumbar region of the spine, between L3 (bony structure 22) and L4 (bony structure 24). In other embodiments, other interspaces may be passed through, including the interspaces in and between the cervical, thoracic and lumbar regions of the spine.

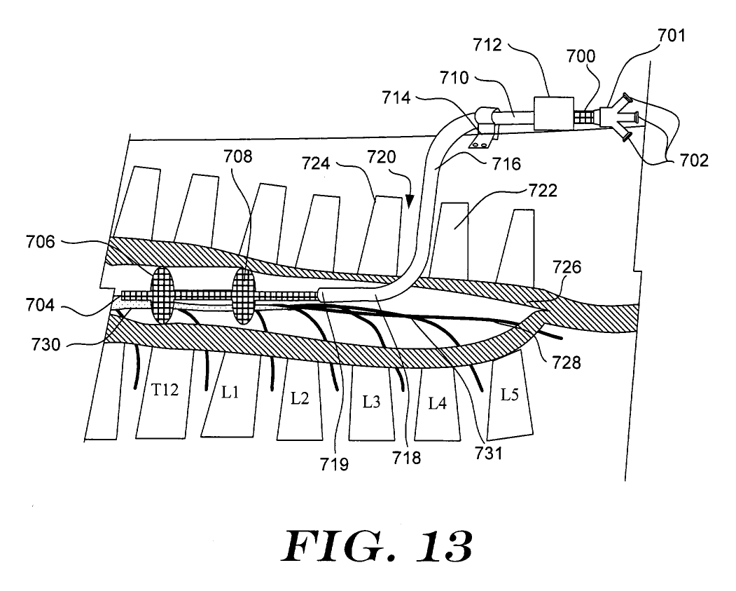

The entry may be performed using a variety of methods. For example, a standard puncture of the spinal subarachnoid space in the lumbar, thoracic or cervical regions, may be performed. A dilator may be used to provide an enhanced opening in some embodiments, as well as an introducer sheath, for example, as presented in co-pending application Ser. No. ______, filed on even date herewith ______, entitled INTRODUCER SHEATH (Attorney docket 1001.1599103) which is expressly incorporated herein by reference. In other embodiments, a device such as the guide catheter 10 may be directly introduced over a puncture needle. FIG. 13, below, illustrates a further embodiment in which an introducer sheath is first inserted.

Included as part of guide catheter 10 is anchoring member 40. The anchoring member 40 is in a deployed position, so that it presses against the edges defining the spinal subarachnoid space 28, which edges may include the dural membrane 26. The illustrative embodiment shown suggests anchoring member 40 as an inflatable device, although in other embodiments other anchoring structures may be used, as illustrated and described below.

To facilitate inflation of anchoring member 40, the guide catheter 10 may include multiple lumens. For the illustrative embodiment of FIG. 1A, the proximal end 12 of guide catheter 10 includes three ports 42, 44, 46. The first port 42 may be used as an entry port for adding contrast fluid, for example, or for insertion of another catheter or other device through the guide catheter 10. The second port 44 may be an inflation port for providing an inflation fluid to the anchoring member 40. The third port 46 may be a port for insertion of a guidewire or other devices. It should be understood that additional or fewer ports and lumens may be used, depending upon the desired capabilities and usage of the device.

FIG. 1B is a cross sectional view of the example embodiment guide catheter 10 of FIG. 1A at a location corresponding to the anchoring member 40. Anchoring member 40 is shown engaging the dural membrane 26 for the illustrative example. In other embodiments, and depending upon the positioning both axially and longitudinally with respect to the spine, the anchoring member 40 may also engage bony structures or any other membrane or structure encountered in or adjacent to the subarachnoid space 28. With the anchoring member 40 engaged with the dural membrane 26, the anchoring member may provide force against movement of the guide catheter in at least one direction, such as, for example, longitudinal direction 50 (FIG. 1A), lateral direction 52, transverse direction 54, or rotational 56 (FIG. 1B).

Anchoring member 40 is illustrated as engaging the dural membrane 26 without pressing the spinal cord 30 against the dural membrane 26. In other embodiments the anchoring member 40 may engage a greater portion of the dural membrane 26, and may conform to most or all of the inner surface of the dural membrane 26, including, perhaps, placing pressure upon the spinal cord 30. Precautions, such as a predefined shape for the anchoring member 40, limits on applied inflation pressure, slow deployment, addition of drugs or medications, use of contrast media to observe structure, or even inducement of a localized hypothermic state in an area near the anchoring member 40 may be taken to reduce the likelihood of causing injury to the spinal cord 30, spinal nerves 31, or dural membrane 26 itself during inflation and while the anchoring member 40 is deployed. Copending application Ser. No. ______ filed on even date herewith ______ entitled METHODS AND APPARATUSES FOR NAVIGATING THE SUBARARCHNOID SPACE (Attorney docket 1001.1599101) discusses methods and apparatuses for inducing a hypothermic condition after accessing the subarachnoid space, and is expressly incorporated herein by reference.

While the example guide catheter 10 illustrated in FIGS. 1A-B is shown advanced within the spinal subarachnoid space 28, in other embodiments, the guide catheter 10 may be advanced to the intracranial subarachnoid space, such that the distal end 14 may navigate around or even through brain tissue and other features contained in the cranium. Such advancement may pass by the foramen magnum as well as the pia mater. Advancement may take place in several methods; in one embodiment, the guidewire 16 is advanced a distance past the distal end 16 of the guide catheter 10, and then guide catheter 10 is advanced over the guidewire 16. In other embodiments, the guide catheter 10 may be advanced without a guidewire 16.

In an illustrative embodiment, the guide catheter 10 having an anchoring device 40 may be used to facilitate passage of the pia mater and entry into the intracranial subarachnoid space. As noted in copending application Ser. No. 09/905,670 filed Jul. 13, 2001 entitled METHODS AND APPARATUSES FOR NAVIGATING THE SUBARACHNOID SPACE, which is incorporated herein by reference, a tough membrane, which may be the pia mater, may be encountered as a device is advanced into the intracranial subarachnoid space, and in order to pierce the tough membrane, pressure may be applied along a stiff device until the resistance offered by the tough membrane is overcome. While force is applied to the stiff device, buckling may occur at some point along its length. Such buckling, if allowed or uncontrolled, could cause damage to the various delicate structures, including spinal nerves and the spinal cord 30, within the subarachnoid space 28. To prevent buckling of the stiff device during such a piercing, a device such as guide catheter 10 may be used to provide anchor points for the stiff device, so that long, unsupported spans of the stiff device are eliminated.

For embodiments such as that illustrated in FIGS. 1A-1B, the anchoring member 40 may be inflated by an inflation fluid. Such inflation fluid may include a variety of materials, including, for example, saline, cerebrospinal fluid, and any other fluid capable of delivering the desired pressure. In some embodiments, the inflation fluid may include radiopaque or other visualization materials to aid in monitoring the shape and location of inflation, for example, if an inflatable member shaped to prevent damage to the spinal cord is used. In some embodiments, the chosen inflation fluid may be adapted for infusion into the subarachnoid space so that, in the event of a rupture or leak of an inflation lumen or inflatable device, the surrounding tissue will not be contaminated with harmful substances. For example, the inflation fluid may be limited in some embodiments to fluids suitable for such introduction.

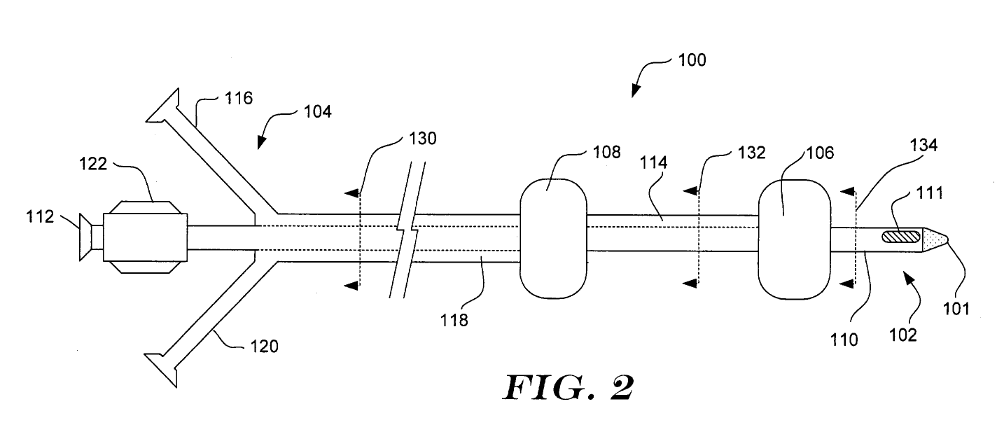

FIG. 2 is a schematic side view of an illustrative embodiment showing a guide catheter 100. Guide catheter 100 includes distal end 102, proximal end member 104, first anchoring member 106, and second anchoring member 108. Distal end 102 extends distally beyond the first anchoring member 106, and distal end 102 may include an exit port 111 and main lumen 110. The distal end 102 may include flexible tip 101 adapted for atraumatic advancement in the subarachnoid space.

Also shown in the diagram is a first inflation lumen 114 and a second inflation lumen 118. The first inflation lumen 114 is in fluid communication with first inflation port 116 as well as first anchoring member 106, while the second inflation lumen 118 is in fluid communication with second inflation port 120 and second anchoring member 108. Main lumen 110 is in fluid communication with main port 112, and may include a non-return valve 122, which may include, for example, a hemostatic valve.

In use, the example guide catheter 100 may be advanced in several ways. In one embodiment, a guidewire (not shown) may be passed through main lumen 110 from main port 112 to distal port 111, and after the guidewire is advanced a distance, the guide catheter 100 may be advanced over the guidewire. In another embodiment, no guidewire is used, and instead the example guide catheter 100 is advanced by itself, with the atraumatic tip 101 providing a safety structure for advancement of the guide catheter 100 around and past sensitive tissue in the subarachnoid space.

As a step of advancement, the first and second anchoring members 106, 108 may be used. For example, the guide catheter may be advanced until the second anchoring member 108 reaches a desired location. Then, inflation fluid may be infused through second inflation lumen 120 to inflate second anchoring member 108, creating an anchored point within the subarachnoid space from which further manipulation of the guide catheter 100 may be performed. Once second anchoring member 108 is placed, the guide catheter 100 may be further manipulated until the first anchoring member 106 is in a desired alignment or location, and the first anchoring member 106 may then be deployed by infusing inflation fluid through first inflation lumen 114.

FIG. 2 shows the two anchoring members 106 and 108 in a substantially extended or inflated state. In a substantially retracted or deflated state, the anchoring members 106, 108 may have a substantially reduced outer diameter, and may lay substantially flat on the outer surface of the guide catheter 100.

With both anchoring members deployed, other devices may be advanced through the main lumen 110 or, for example, fluid may be infused through main lumen 110 to a desired location. In another example, an infusion catheter may be advanced through main lumen 110 leaving enough open space in main lumen 110 to allow fluid drainage through main lumen 110, so that while fluid is infused by the infusion catheter, fluid may also be drained through main lumen 110. Pressures created at the distal tip, for example, by fluid infusion, may be prevented from moving the guide catheter 100 by the anchoring members 106, 108. Also, other devices or catheters advanced through main lumen 110 may be advanced and withdrawn more quickly than if the other devices or catheters had to carefully traverse the subarachnoid space itself. To ease advancement, main lumen 110 may include a lubricious inner coating. Thus, the guide catheter 100 may provide an anchored, stable pathway for entry while also protecting adjacent tissue from devices advanced therethrough.

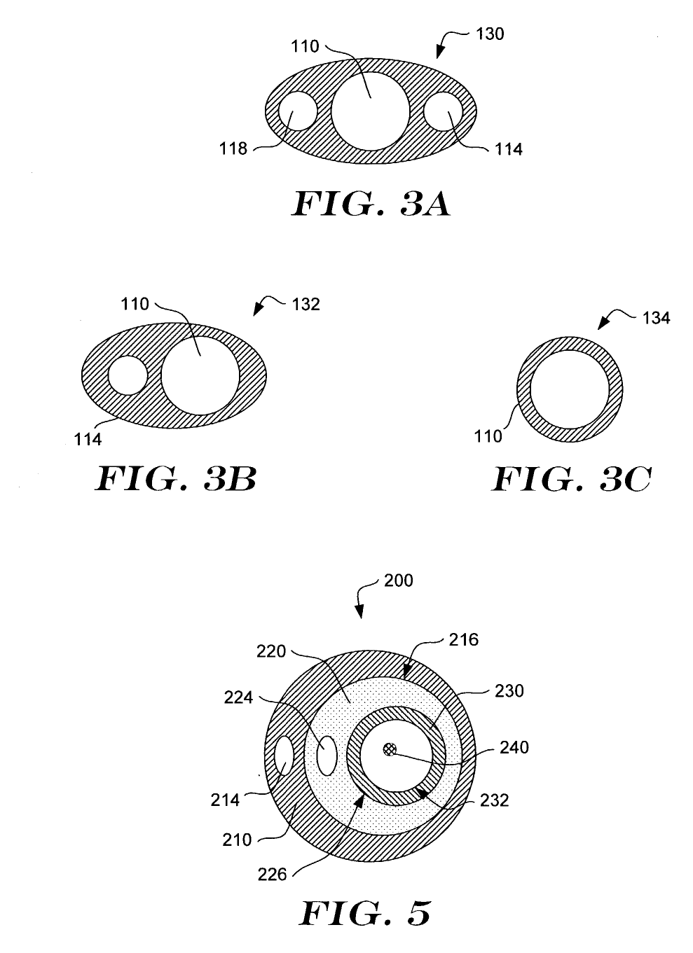

FIGS. 3A-3C are cross sectional views of portions of the example guide catheter 100 shown in FIG. 2. For example, FIG. 3A corresponds to a cross sectional view of the portion of guide catheter 100 at location 130. The configuration shown includes first inflation lumen 114, second inflation lumen 118, and main lumen 110. While the exact proportions may vary in other embodiments, for the embodiment shown the main lumen 110 may be the largest of the three. The side by side arrangement shown in FIG. 3A is merely one method of providing the multiple lumens; for example other illustrative embodiments may use a coaxial arrangement, and a hybrid arrangement including multiple slidably disposed side-by side catheters in what could also be described as an off-set coaxial configuration is shown in FIG. 5.

FIG. 3B corresponds to a cross sectional view of the portion of guide catheter 100 at location 132. It may include, again, inflation lumen 114 and main lumen 110. Notably, because location 132 is distal the second anchoring member 108, the second inflation lumen 118 is not included, since it terminates adjacent the second anchoring member 108.

Without second inflation lumen 118, the guide catheter 100 portion near location 132 may have a different flexibility than the portion near more proximal location 130. In some illustrative embodiments, the portion near location 132 may be made of a stiffer material than the more proximal portion near location 130, but may have less cross sectional area so that the overall stiffness does not vary. For example, a braided support member having a varying density (crossings per inch, for example) or other such support member may be included. In another embodiment, each portion is made of materials possessing similar qualities, but because the guide catheter 100 cross section becomes smaller, the resulting stiffness decreases from proximal locations to distal locations. Other embodiments encompass further variations. Notably, the guide catheter 100 may be similarly composed throughout, or may have varying material compositions at different locations.

FIG. 3C corresponds to a cross sectional view of the portion of guide catheter 100 at location 134. At this even more distal location 134, both inflation lumens 114, 118 are eliminated, as both may terminate adjacent corresponding anchoring members 106, 108. Main lumen 110 may extend beyond both anchoring members, as illustrated.

In other embodiments, the inflation lumens 114, 118 may be extended a further distance, for example, to reduce fabrication costs, to provide consistency throughout the length of guide catheter 100, or in one example embodiment, to allow for increased stiffness throughout the guide catheter once the lumens 114, 118 are under pressure. Thus, for example, once the anchoring members 106, 108 are inflated, the inflation lumens 114, 118 could provide support to the guide catheter 100 to increase stiffness.

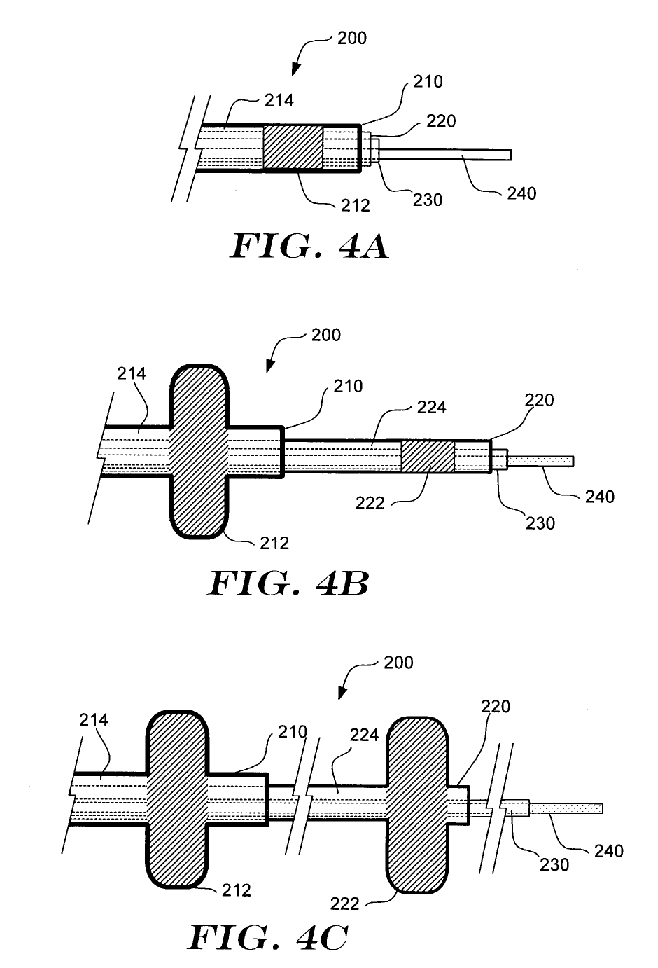

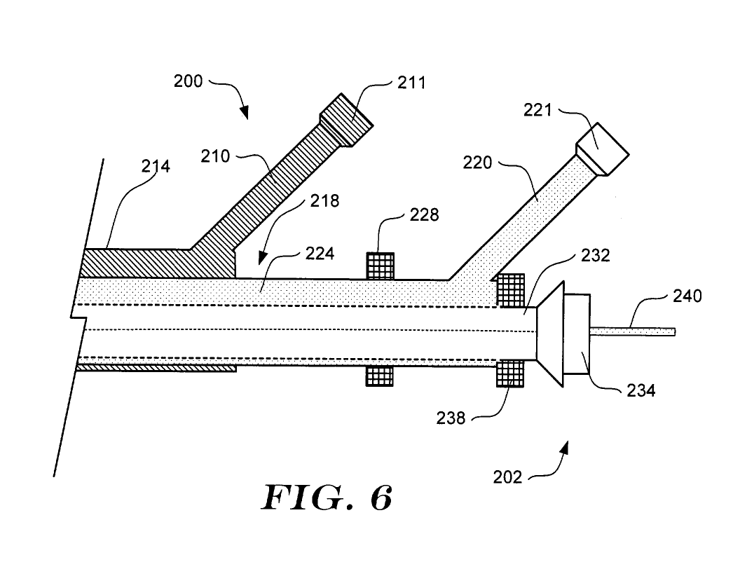

FIGS. 4A-4C are schematic side views of an illustrative embodiment showing a guide catheter 200 in several stages of placement. FIGS. 4A-4C are best understood with additional reference to FIGS. 5 and 6, in which like elements are numbered the same. FIG. 5 is a cross sectional view of guide catheter 200 illustrating an example configuration for the multiple elements that make up guide catheter 200. FIG. 6 is a schematic side view that illustrates an example proximal end 202 for guide catheter 200.

FIG. 4A illustrates a guide catheter 200 having a first element 210, a second element 220, a third element 230 and a guidewire 240. First element 210 includes a first anchoring member 212 and a first inflation lumen 214. FIG. 5 illustrates a cross section of guide catheter 200, and shows that first inflation lumen 214 may be integrated as part of first element 210 in a side by side configuration with a main lumen 216 of first element 210. Main lumen 216 and second element 220 may be sized as shown in FIG. 5 to allow second element 220 to be slidingly disposed within first main lumen 216. As shown in FIG. 6; the first inflation lumen 214 may be connected to first port 211 of first element 210. First port 211 may include, for example, a non-return valve, a Leur lock, or a hemostatic valve.

FIG. 4B illustrates a step in advancement of guide catheter 200. After the first anchoring member 212 is advanced, to a desired location, the first anchoring member 212 may be expanded by providing inflation fluid through first port 211 (FIG. 6) into the first inflation lumen 214. After the first anchoring member 212 is inflated, the first element 210 may be anchored by the first anchoring member 212. Then the rest of the guide catheter 200 may be advanced, since the second element 220 may be slidably disposed within the first main lumen 216 of the first element 210. Further, as illustrated in FIG. 5, the third element 230 may be slidably disposed within the main lumen 226 of second element 220 and guidewire 240 may be slidably disposed within the lumen 232 of the third element 230. Thus the second element 220 and third element 230 as well as the guidewire 240 may be advanced after the first anchoring member 212 is expanded by inflation.

Also shown in FIG. 4B is additional detail of the second element 220. Second element 220 may include a second anchoring member 222 as well as an inflation lumen 224, which is also illustrated in FIG. 5. As shown in FIG. 6, the second element 220 may include a second port 221 near proximal end 202. The second port 221 may be in fluid communication with the inflation lumen 224. An additional feature shown in FIG. 6 is first stop 228. First stop 228 may be used to prevent damage to second port 221 as the second element is advanced to near its limit at the location of first port 211. When stop 228 comes into contact with the joint 218 where first port 211 extends outward, stop 228 prevents further advancement. Though stop 228 is explicitly included in some embodiments, in other embodiments it may be omitted.

FIG. 4C illustrates a further step in the example advancement with the guide catheter 200. As shown, second anchoring member 222 has been inflated with fluid infused through second port 221 and through second inflation lumen 224. Thus, a second anchoring location is defined for the guide catheter 200, this one more distal than the first anchoring location defined by the first anchoring member 212. By use of the second anchoring member 222, the second element 220 may be anchored in place. Third element 230 defining third main lumen 232 may then be advanced as before, since it is slidably disposed within the second main lumen 226.

The third element 230 may include a valve apparatus 234 at the proximal end 202 (FIG. 6). The valve apparatus 234 may be adapted to allow various devices, such as catheters, guidewires, endoscopes and the like to be passed therethrough, as well as, for example, allowing a fluid to be infused or drained thereby. While omitted in some embodiments, guidewire 240 may be used to aid in the advancement of the guide catheter 200. Overall, the illustrative example shown in FIGS. 4A-4C may be described, generally, as representing an articulating guide catheter.

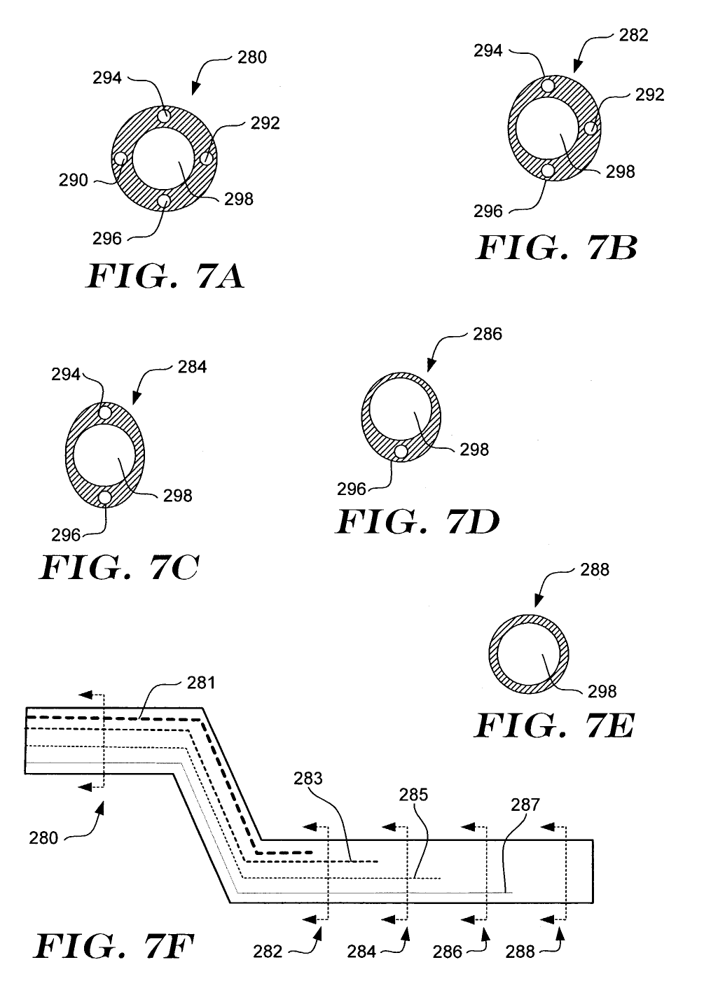

FIGS. 7A-7E are cross sectional views of sections of an illustrative guide catheter. The catheter may include several segments along its length, each depicted by one of the views 7A-7E. For example, a most proximal portion 280 may include five lumens: four ancillary lumens that may include first lumen 290, second lumen 292, third lumen 294, and fourth lumen 296, along with a main lumen 298. In some embodiments, each of the four ancillary lumens 290, 292, 294, 296 may be inflation lumens, for use to inflate anchoring members, for example, as shown in FIGS. 4A-4C.

FIG. 7F is a schematic side view of an illustrative guide catheter corresponding to the example embodiment illustrated throughout FIGS. 7A-7E. The various views shown in FIGS. 7A-7E may be noted with reference to FIG. 7F. Included in FIG. 7F are four shaping members 281, 283, 285, 287, varying in stiffness as suggested by their varying thicknesses, with first shaping member 281 the stiffest and fourth shaping member 287 the most pliable. Each of the four shaping members 281, 283, 285, 287 may have the property that each is stiffer at a first temperature than when at a second temperature. Note that the illustrative guide catheter adopts a shape resembling an “S,” as suggested in copending patent application Ser. No. ______ filed even date herewith ______ entitled INTRODUCER SHEATH (Attorney Docket 1001.1599.103), which is expressly incorporated herein by reference.

The guide catheter is designed so that the first lumen 290 terminates at a first-most proximal location, so the guide catheter takes on the cross section FIG. 7B, including second lumen 292, third lumen 294, fourth lumen 296, and main lumen 298, for example at location 282. Likewise, second lumen 292 terminates at a second-most proximal location, so the guide catheter takes on the cross section shown in FIG. 7C, including third lumen 294, fourth lumen 296, and main lumen 298, at another location 284. Again, the third lumen 294 terminates at a third-most proximal location, so the guide catheter takes on the cross section shown in FIG. 7D, including fourth lumen 296 and main lumen 298, for example at location 286. Finally, the fourth lumen terminates, leaving only main lumen 298, for example, at location 288 and as shown in FIG. 7E. In some embodiments, each of the ancillary lumens may be used to contain a shaping member 281, 283, 285, 287. For example, an elongate shaping member 281 sized to slidably fit within first lumen 290 may be inserted along the length of the first lumen 290 after the guide catheter, at least for the length of first lumen 290, has been advanced to a desired location or alignment. The shaping member 281 may be designed to be pliable when heated and stiff when cooled, for example. Then, after the shaping member 281 is heated to be pliable, it is inserted into the first lumen 290 until the distal end of the shaping member 281 reaches the point of termination of the first lumen. Once inserted fully, the shaping member 281 is allowed to cool (for example, the “heated” temperature may be in the range of about one hundred and thirty degrees Fahrenheit, and the “cooled” temperature may be in the range of about 98.6 degrees Fahrenheit, for use in a human subarachnoid space) and become relatively stiffer. Thus, a chosen location within the subarachnoid space may be accessed, and the guide catheter 280 stiffened so it retains a shape corresponding to such access. The process may be repeated for each of the four lumens, until four shaping members 281, 283, 285, 287 are inserted and the guide catheter becomes rigidly held in place merely by its shape.

In one such embodiment, the shortest shaping member 281 is the stiffest, with each successive shaping member 283, 285, 287 less stiff. Once the entire guide catheter with all shaping members 281, 283, 285, 287 is inserted, a diagnostic or therapeutic procedure, for example, may be performed. After completion of the procedure, the stiffest shaping member 281 may be removed first, with the other three remaining shaping members 283, 285, 287 used to provide tension against damaging surrounding tissue during extraction. As successively more flexible shaping members 283, 285, 287 are removed, the remaining shaping members 283, 285, 287 are used to provide protective tension, until a relatively stiff guidewire is used within the main lumen 298 to provide protective tension during extraction of the last remaining shaping member 287.

It should be understood that more or fewer lumens and shaping members may be used in other embodiments. The number of lumens and shaping members may be determined based on the desired properties and intended shape and/or size of the device. Furthermore, additional lumens to the main lumen 298 which do not include the shaping members may be provided.

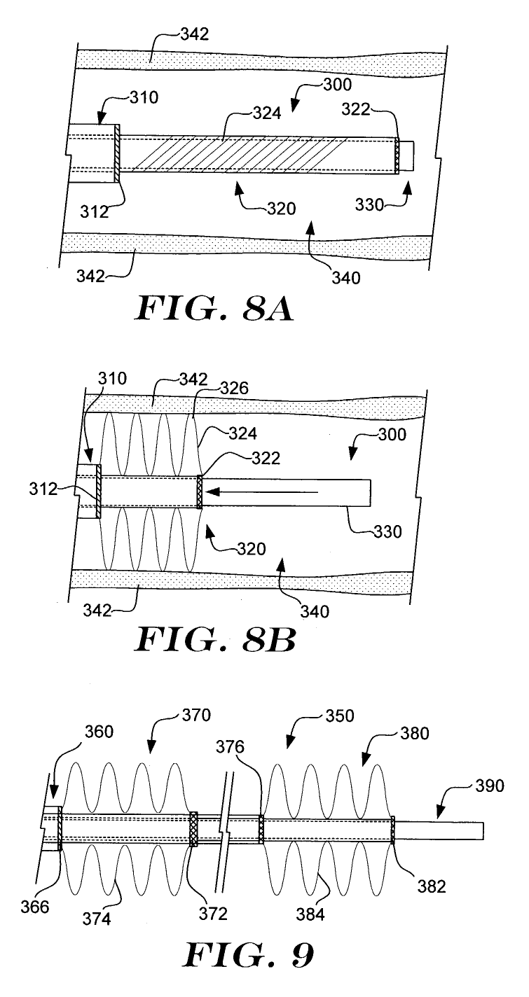

FIGS. 8A-8B are schematic side views of an illustrative guide catheter including an anchoring member in retracted and deployed positions. The guide catheter 300 includes a first element 310, second element 320, and third element 330. Guide catheter 300 is shown advanced into subarachnoid space 340 having defining membrane 342.

The third element 330 is slidably disposed within the second element 320, which is in turn slidably disposed within the first element 310. Included between the distal end 322 of the second element 320 and the distal end 312 of the first element 310 is anchoring member 324. As shown in FIG. 8A, the anchoring member 324 is fittingly disposed as part of the second element 320. As shown in FIG. 8B, the relative distance between distal end 322 of the second element 320 and the distal end 312 of the first element 310 is reduced, causing the anchoring member 324 to wrinkle or fold up, expanding outward. The outward expansion creates an anchoring point once the outermost portions 326 of the anchoring member 324 engage membrane 342 which defines the subarachnoid space 340 into which the guide catheter 300 is advanced. These movements may be manipulated from a proximal end of the guide catheter 300, for example, by holding the first element 310 in position while pulling on the second element 320. A locking mechanism may be included to hold the first element 310 and second element 320 in a desired configuration, for example, keeping the anchoring member 324 in either the withdrawn position shown in FIG. 8A or the expanded position shown in FIG. 8B.

In an illustrative embodiment, the anchoring member 324 may be made of a relatively flexible or soft material, but may include portions of greater stiffness, for example, to provide a particular shape to the anchoring member 324 as it is deployed. The anchoring member 324 may be any suitable structure, including, for example, a serrated section, a braid structure, an expandable woven section, or an elastomeric member, for example, or any other structure that expands when compressed. Alternatively, anchoring member 324 may be any structure that will radially contract when stretched, so that the anchoring member may be inserted to the subarachnoid space in a stretched state, for example subject to a longitudinal force, and then relaxed or released by the relative motion of distal ends 312, 322 toward each other.

It should be understood that, for each of these illustrative embodiments, the step of an anchoring member 324 engaging a membrane 342 is purely illustrative of one way in which an anchoring member 324 may perform an anchoring function. An anchoring function may be performed by providing for resistance against the CSF filling the subarachnoid space 340, for example, or against bony structures, spinal nerves, the spinal cord, or other tissues therein. While it should be understood: that engaging, for example, the spinal cord itself, may present risk of damage to the spinal cord, such risks may be measured against the potential benefits of an operation, and may be mitigated, for example, by limiting the extent of such engagement, by providing for engagement over a large area or at many locations, or by providing an anchoring function in limited directions defined to minimize potential damage to tissue used to secure such anchoring functions.

FIG. 9 is a schematic side view of an illustrative guide catheter having multiple anchoring members. The guide catheter 350 may include a first element 360, a second element 370, a third element 380, and a fourth element 390, the second element 370 slidingly disposed within a lumen in the first element 360, the third element 380 slidingly disposed within a lumen in the second element 370, and the fourth element 390 slidingly disposed within a lumen in the third element 380.

The first element 360 may include first distal element 366, which can provide a connection against first anchoring member 374 that also connects to collar 372 on second element 370. The second element 370 may also include a second distal end 376 separated by some distance from the collar 372. The second distal end 376 may connect to second anchoring member 384, which in turn connects to the third element 380 including third distal end 382. The fourth element 390 may extend beyond the third distal end 382. In operation, each anchoring member 374, 384 may be expanded at a desired location to provide further guiding and anchoring for the overall guide catheter 350 in a manner similar to that noted above with respect to FIGS. 8A-8B. Note that each of the elements 360, 370, 380, 390 may be designed so that, as shown in FIG. 8C, the anchoring members are not at the distal end of the elements, which may facilitate easier introduction of the guide catheter 350 by reducing relative change of diameter at the distal ends of each element (sometimes referred to as a shoulder), thus reducing the “shoulder” caused by transitions from element to element and limiting tissue trauma caused by advancement of the guide catheter.

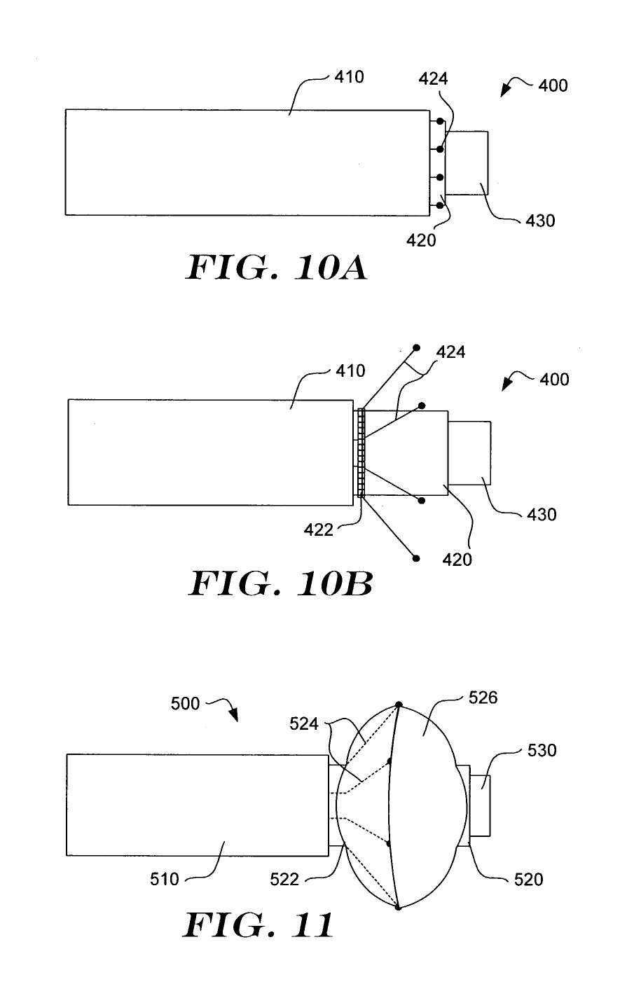

FIGS. 10A-10B are schematic side views of another illustrative guide catheter including an anchoring mechanism in retracted and deployed positions. The guide catheter 400 may include a first element 410, a second element 420, and a third element 430. The second element 420 may include one or more anchor members 424. As shown in FIG. 10A, the first element 410 may cover part (or even all) of the anchor members 424 when in a first position with respect to the second element 420.

As shown in FIG. 10B, the first element 410 may be retracted, uncovering anchor members 424 and collar 422. Collar 422 may hold the anchor members 424 in place with respect to the second element 420 and may provide a base for tension in the anchor members 424. In other embodiments, collar 422 may be excluded and the anchor members 424 may be simply attached directly to the second element 420.

In operation of the illustrative embodiment, the anchor members 424 may be disposed under tension when covered by the first element 410. Once the guide catheter 400 is advanced to a desired location within the subarachnoid space, anchor members 424 may expand or spring outward as the first element 410 is withdrawn, engaging surrounding tissue (not shown). Once so expanded and engaged, the anchor members 424 can provide an anchoring point for the guide catheter 400, and the third element 430 may extend therefrom. For example, the third element 430 may be slidingly disposed within the second element 420 by, for example, coating the third element 430, an inner lumen of the second element 420, or both, with a lubricious material. To remove the anchored guide catheter 400, the first element 410 may be advanced to cover and restrain the anchoring members 424, so the catheter 400 comes to resemble that shown in FIG. 10A once again.

Anchor members 424 may be formed, for example, of bent wires or pins, in some embodiments. In another embodiment, anchor members 424 may be formed of a spring coil. Likewise, anchor members 424, rather than extending outward along the longitudinal direction of guide catheter 400, as shown, may instead extend in an axial direction, in effect spiraling outward. Also, in another embodiment, rather than retracting a first element 410 to release tension in anchor members 424, a solder ring, for example, may be pulled beneath the anchor members 424 to force anchor members 424 outward.

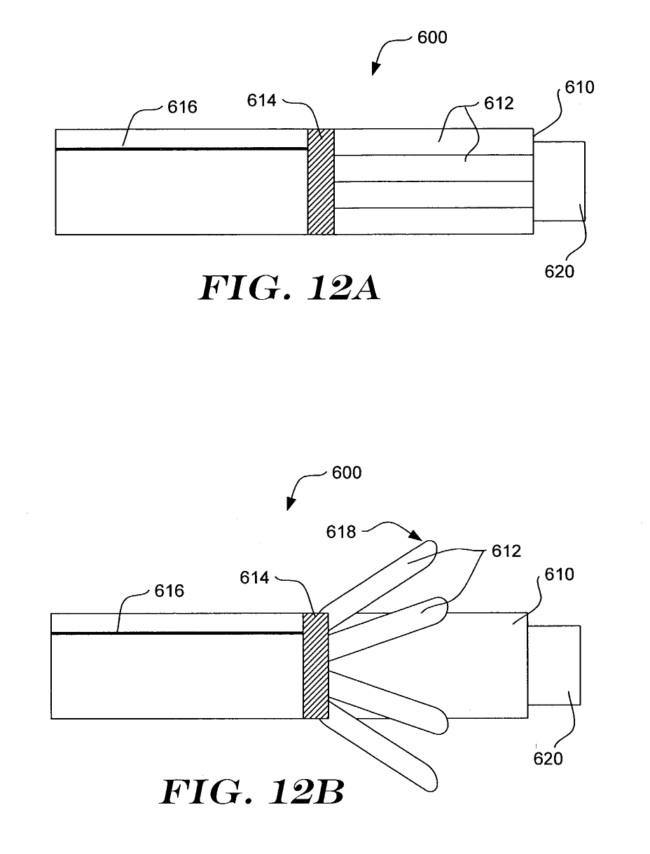

FIG. 11 is a schematic side view of another illustrative guide catheter with an anchoring member covered by a membrane. The guide catheter 500 includes a first element 510, a second element 520, and a third element 530. The guide catheter 500 in many respects may be similar to that shown in FIGS. 10A-10B, except for the addition of a membrane 526 that covers anchoring members 524. As before, the anchoring members 524 may expand outward from collar 522 as first element 510 is retracted after insertion into a subarachnoid space. The illustrative embodiment of FIG. 11 may have an advantage insofar as the membrane 526 can prevent the anchoring members 524 from becoming tangled with surrounding tissue fibers, such as spinal nerves extending outward from the spinal cord, or with other tissue, for example, tissue found in the intracranial subarachnoid space.

FIGS. 12A-12B are schematic side views of another illustrative guide catheter including a shape memory anchoring mechanism. Illustrative guide catheter 600 is shown including a first element 610 and a second element 620. The second element 620 may be slidingly disposed within the first element 610. One or more anchoring members 612 are shown connected to a collar 614. The collar 614 may in turn connect to heat conducting element 616.

As shown in FIG. 12A, the anchoring members 612 are in a collapsed state, holding closely to the first element 610. The anchoring members 612 may have shape memory properties so that, for example, when exposed to a change in temperature they spring outward as shown in FIG. 12B. Each of the anchoring members 612 may include an atraumatic tip 618 devised for atraumatically engaging a surrounding membrane (not shown).

The anchoring members 612 may, for example, comprise thin strips of metal, rods, pins, or other shapes. In one example, the anchoring members 612 may include two different materials having different coefficients of thermal expansion, adhered to one another so that, when a temperature change is induced to the overall anchoring member 612, one material expands more than the other, inducing curvature of the anchoring member 612.

In one embodiment, the conducting element 616 includes wires for conducting electricity through a resistive element, for example, included in collar 614, heating at least a portion of the anchoring members 612. In another embodiment, conducting element 616 may include two lumens, one an inlet and another an outlet, for circulating a cooling or heating fluid through collar 614, again to induce a temperature change in a portion of the anchoring members 612 to actuate the shape memory action. In other embodiments, the conducting element may be any sort of element that can induce a temperature change for selectively actuating the shape memory action of the anchoring members 612.

Each of these non-inflating anchoring members shown or described in relation to FIGS. 7-12 may be substituted for the inflatable anchoring members shown and described in relation to FIGS. 1-6, with appropriate modifications to the guide catheters shown therein where needed. Further, multiple members may be included in a single device, and these various different configurations may be readily mixed so that, for example, an inflatable member as shown in FIG. 4 may be used with a shape memory member as shown in FIG. 12 along different locations or along different axes of the same guide catheter.

FIG. 13 is a diagrammatic side view of an entry into a spinal subarachnoid space with an example guide catheter having an anchoring member and including an entry sheath. Guide catheter 700 includes proximal end member 701 having several ports 702, along with distal end 704. Along the length of guide catheter 700 are first anchoring member 706 and second anchoring member 708.

Also shown is introducer sheath 710 having a proximal end member 712, attachment apparatus 714, intermediate section 716, distal section 718 and distal port 719. The guide catheter 700 is shown passing through a lumen in the introducer sheath 710, entering through the proximal end member 712 and exiting at distal port 719. The introducer sheath 710 may, for example, be similar to those suggested in copending patent application Ser. No. ______ filed on even date herewith ______ entitled INTRODUCER SHEATH (Attorney docket 1001.1599103), which is incorporated herein by reference.

The introducer sheath 710 assists in introducing the guide catheter 700 into subarachnoid space 728 by passing through interspace 720 between bony structures 722, 724 and through dural membrane 726. The introducer sheath 710 may be introduced to the subarachnoid space 728 first, with the guide catheter 700 advanced through the introducer sheath once the distal port 719 is advanced to a first desired position. Then, the guide catheter 700 may be advanced from the distal port 719 to a second desired position where second anchoring member 708 may be expanded or actuated to engage the dural membrane 726. The second anchoring member 708 may also engage spinal cord 730, or it may be adapted to avoid applying pressure to or engaging the spinal cord 730.

After the second anchoring member 708 is engaged with dural membrane 726, the guide catheter 700 may be further manipulated, for example, until first anchoring member 706 gains a third desired position. Once at the third desired position, the first anchoring member 706 may then be expanded or actuated to engage the dural membrane 726. Once both anchoring members 706, 708 are in place and engaged with the dural membrane 726, a secure guide is created by the guide catheter 700. The secure guide may be used, for example, to allow the guide catheter 700 to be used in a diagnostic or therapeutic procedures such as fluid flushing, drainage, infusion or exchange, ablation of tissue, localized cooling, pressure or temperature monitoring, visualization procedures, etc. In other embodiments, additional devices may be advanced through the guide catheter 700 to perform such procedures.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A medical device having a distal end, a proximal end, and an elongate member therebetween, the medical device comprising:
a first inflatable member, the inflatable member disposed on the elongate member such that, when inflated, the inflatable member extends outward from the elongate member, the first inflatable member adapted to expand to a size corresponding to an inner dimension of a patient's subarachnoid space; and
a first inflation lumen providing fluid communication with the first inflatable member from a location proximal of the first inflatable member.

2. The medical device of claim 1, further comprising a passage lumen extending through the elongate member between a location proximal the first inflatable member and a location distal the first inflatable member, the passage lumen sized to slidably pass at least a guidewire therethrough.

3. The medical device of claim 1, further comprising:
a second inflatable member disposed along the elongate member at a location between the first inflatable member and the distal end of the medical device;
a second inflation lumen providing fluid communication with the second inflatable member from a location proximal of the first inflatable member; and
a passage lumen extending within the elongate member between a location proximal the first inflatable member and a location distal the second inflatable member.

4. The medical device of claim 3, wherein at least one of the first inflatable member and the second inflatable member is adapted to expand to a size corresponding to an inner dimension of a patient's spinal subarachnoid space.

5. The medical device of claim 3, wherein at least one of the first inflatable member and the second inflatable member is adapted to limit motion along a direction corresponding to the elongate axis of the elongate member when disposed at a location within a patient's spinal subarachnoid space.

6. The medical device of claim 1, wherein the first inflatable member is adapted to expand in such a way as to avoid damaging tissue within a patient's subarachnoid space.

7. The medical device of claim 1, wherein the first inflatable member is adapted to limit motion along a direction corresponding to the elongate axis of the elongate member when disposed at a location within a patient's spinal subarachnoid space.

8. The medical device of claim 1, wherein the first inflatable member is adapted to limit motion along a direction perpendicular to the elongate axis of the elongate member when disposed at a location within a patient's spinal subarachnoid space.

9. The medical device of claim 1, wherein the medical device is a catheter.

10. The medical device of claim 1, wherein the medical device is a guide catheter.

11. The medical device of claim 1, wherein the patient is human.

12. A medical device having a proximal end and a distal end and an elongate member therebetween, the medical device comprising:
a first lumen;
a second lumen; and
a first shapable member having a first stiffness when at a first temperature and a second stiffness when at a second temperature, the first stiffness being greater than the second stiffness;
wherein the shapable member is adapted for insertion into the first lumen and wherein the first temperature corresponds to a patient's normal body temperature.

13. The medical device of claim 12, wherein the elongate shaft is adapted for insertion into the patient's spinal subarachnoid space, and wherein, when the shapable member is disposed at least partially in the first lumen while the elongate shaft is in the spinal subarachnoid space, the elongate shaft is more difficult to move than when the shapable member is not disposed in the first lumen.

14. The medical device of claim 12, further comprising:
a third lumen; and
a second shapable member adapted for insertion into to the third lumen, the second shapable member having a third stiffness greater than the first stiffness when at the first temperature and a fourth stiffness less than the third stiffness when at a third temperature.

15. The medical device of claim 12, wherein the medical device is a catheter.

16. The medical device of claim 12, wherein the medical device is a guide catheter.

17. A medical device having a proximal end and a distal end, the medical device comprising:
a first component including a first elongate member defining a first lumen, and a first anchoring device; and
a second component including a second elongate member defining a second lumen and a second anchoring device;
wherein the second lumen and the first component are adapted to allow the first component to be slidably disposed within the second component for at least a portion of the first elongate member.

18. The medical device of claim 17, wherein at least one of the first anchoring device and the second anchoring device is adapted to provide an anchoring function within a patient's spinal subarachnoid space.

19. The medical device of claim 17, wherein the first anchoring device includes a shape memory member.

20. The medical device of claim 17, wherein the first anchoring device includes a folding member.

21. The medical device of claim 17, wherein the medical device is a catheter.

22. The medical device of claim 17, wherein the medical device is a guide catheter.

23. A medical device having a proximal end and a distal end, the medical device comprising:
a first component including a first elongate member defining a first inflation lumen, a first inflatable member in fluid communication with the first inflation lumen, and a first passage lumen therethrough; and
a second component including a second elongate member defining a second inflation lumen, a second inflatable member in fluid communication with the second inflation lumen, and a second passage lumen therethrough;
wherein the second passage lumen and the first component are adapted to allow the first component to be slidably disposed within the second component for at least a portion of the first elongate member.

24. The medical device of claim 23, wherein the second component is adapted for insertion into a patient's subarachnoid space.

25. The medical device of claim 23, wherein the first inflatable member is adapted for inflation within a patient's subarachnoid space.

26. The medical device of claim 23, wherein the medical device is a catheter.

27. The medical device of claim 23, wherein the medical device is a guide catheter.

28. A method of positioning a device inside a subarachnoid space, the method comprising:
providing a medical device including an anchoring member, the anchoring member having a first configuration and a second configuration, wherein when the anchoring member is disposed in the first configuration the anchoring member is easier to move within the subarachnoid space than when the anchoring member is disposed in the second configuration;
introducing the medical device into a spinal subarachnoid space within the subarachnoid space with the anchoring member in the first configuration;
advancing the medical device until the anchoring member is in a desired position; and
causing the anchoring member to be disposed in the second configuration once the anchoring member is in the desired position.

29. The method of claim 28, wherein the medical device includes a lumen extending from a location proximal the anchoring member to a location distal the anchoring member, the method further including the step of introducing a device through the lumen from a proximal end of the lumen to a distal end of the lumen.

30. The method of claim 28, wherein the anchoring member includes an inflatable device, and the step of causing the anchoring member to be disposed in the second position includes inflating the inflatable device.

31. The method of claim 30, wherein the step of inflating the inflatable device includes providing a pressurized fluid including cerebrospinal fluid.

32. The method of claim 30, wherein the anchoring member includes a shape memory device, and the step of causing the anchoring member to be disposed in the second position includes heating the shape memory device.

33. The method of claim 30, wherein the anchoring member includes a shape memory device, and the step of causing the anchoring member to be disposed in the second position includes cooling the shape memory device.

34. The method of claim 28, wherein the medical device is a catheter.

35. A guide catheter, the guide catheter comprising:
a first elongate member having a proximal end and a distal end and including a first anchoring means for effecting an anchoring function; and
a second elongate member having a proximal end and a distal end and including a second anchoring means for effecting an anchoring function;
wherein the first elongate member and the second elongate member are engaged for at least a portion of the length of the first elongate member, and wherein the second elongate member is adapted to slide beyond the distal end of the first elongate member.

36. The guide catheter of claim 35, wherein the first elongate member and the second elongate member are adapted to construct an articulating member.

37. The guide catheter of claim 35, wherein the first anchoring means is adapted to provide an anchoring function within a patient's subarachnoid space.

38. The guide catheter of claim 37, wherein the anchoring function is operable in a direction perpendicular to the elongate direction of the first elongate member.

39. The guide catheter of claim 37, wherein the anchoring function is operable in a direction parallel to the elongate direction of the first elongate member.

40. The guide catheter of claim 35, wherein the second anchoring means is adapted to provide an anchoring function within a patient's subarachnoid space.

41. The guide catheter of claim 40, wherein the anchoring function is operable in a direction perpendicular to the elongate direction of the second elongate member.

42. The guide catheter of claim 40, wherein the anchoring function is operable in a direction parallel to the elongate direction of the second elongate member.

43. The guide catheter of claim 35, wherein the first anchoring means includes an anchoring device not included in the second anchoring means.

44. The guide catheter of claim 35, wherein at least a portion of the second elongate member is disposed within a portion of the first elongate member.

45. A method of navigating a patient's subarachnoid space, the method comprising:
providing a navigation device having a proximal end and a distal end and including a first anchoring device and a second anchoring device;
percutaneously introducing the navigation device into the spinal subarachnoid space;
manipulating the navigation device into a first desired position within the subarachnoid space;
causing the first anchoring device to perform an anchoring function;
manipulating the navigation device into a second desired position within the subarachnoid space; and
causing the second anchoring device to perform an anchoring function.

46. The method of claim 45, wherein the step of manipulating the navigation device into a first desired position includes advancing the distal end of the navigation device toward the patient's head.

47. The method of claim 45, wherein the step of manipulating the navigation device into a first desired position includes controlling the direction of the distal end of the navigation device with respect to a longitudinal direction of the patient's spine at a location corresponding to the first anchoring device.

48. The method of claim 45, wherein the step of manipulating the navigation device into a second desired position includes advancing the distal end of the navigation device toward the patient's head.

49. The method of claim 45, wherein the step of manipulating the navigation device into a second desired position includes controlling the direction of the navigation device with respect to a longitudinal direction of the patient's spine at a location corresponding to the second anchoring device.

50. The method of claim 45, wherein the distance between the first anchoring device and the second anchoring device may be varied.

51. The method of claim 50, further including the step of varying the distance between the first anchoring device and the second anchoring device after causing the first anchoring device to perform an anchoring function.

52. The method of claim 45, wherein the distance between the first anchoring device and the distal end of the navigation device may be varied.

53. The method of claim 52, further including the step of varying the distance between the first anchoring device and the distal end of the navigation device after causing the first anchoring device to perform an anchoring function.

54. The method of claim 45, wherein the distance between the second anchoring device and the distal end of the navigation device may be varied.

55. The method of claim 54, further including the step of varying the distance between the second anchoring device and the distal end of the navigation device after causing the second anchoring device to perform an anchoring function.

56. A medical device having a distal end, a proximal end, and an elongate member therebetween, the medical device comprising:
an anchoring device, the anchoring device engaging the elongate member such that the anchoring device may selectively perform an anchoring function when a portion of the elongate member engaged with the anchoring device is disposed within a spinal subarachnoid space; and
an actuation apparatus for enabling the selectivity of the first anchoring device.

57. The medical device of claim 56, wherein the anchoring device includes an inflatable member and the actuation apparatus includes an inflation lumen in fluid communication with the inflatable member, the inflation lumen being externally accessible when the portion of the elongate member engaged with the anchoring device is disposed within the spinal subarachnoid space.

58. The medical device of claim 56, wherein the anchoring device includes a shape memory material adapted for use within the spinal subarachnoid space, and the actuation apparatus includes a heat transfer device that can cause the shape memory material to change shape.

59. The medical device of claim 56, wherein the anchoring device includes an expandable member and the actuation apparatus is adapted to cause the expandable member to selectively expand and contract.

60. The medical device of claim 56, wherein the anchoring device includes an array of engaging members having a predefined shape and elastic properties, wherein the actuation apparatus includes an outer member slidably disposed over the elongate member, wherein when the actuation apparatus is in a first position, the array of engaging members is held under tension between the elongate member and the outer member and when the actuation apparatus is in a second position, at least one of the array of engaging members engages a feature of the spinal subarachnoid space.

61. The medical device of claim 56, wherein the anchoring device includes an expandable braid which increases in diameter when longitudinally compressed.

62. The medical device of claim 56, wherein the anchoring device includes an expandable braid which reduces in diameter when longitudinally stretched.

63. The medical device of claim 56, wherein the medical device comprises a catheter.

64. The medical device of claim 56, wherein the medical device comprises a guide catheter.

65. A balloon catheter inflation system comprising:
a catheter having a proximal end, a distal end, an elongate section therebetween, the elongate section including an inflation lumen therethrough and an inflatable member disposed thereupon; and
an inflation fluid comprising materials adapted for introduction into the spinal subarachnoid space.

66. The balloon catheter inflation system of claim 65, wherein the inflation fluid comprises cerebrospinal fluid.

67. The balloon catheter inflation system of claim 65, wherein the inflation fluid consists of materials and fluids suitable for introduction into the subarachnoid space.

68. The balloon catheter inflation system of claim 65, wherein the inflatable member is adapted for use in a subarachnoid space.

69. A medical device comprising:
an elongate member including a proximal end and a distal end;
a means for anchoring a portion of the elongate member within a subarachnoid space of a patient.

70. A method of navigating a patient's subarachnoid space, the method comprising:
providing an elongated navigation device including a means for anchoring a portion of the navigation device within the subarachnoid space;
percutaneously introducing the portion of the navigation device within the subarachnoid space; and
anchoring the portion of the navigation device within the subarachnoid space using the means for anchoring.
